(19) United States
(12) Plant Patent Application Publication
(10) Pub. No.: US 2002/0073453 P1
(43) Pub. Date: Jun. 13, 2002

(54) New plant of abelia X grandiflora named 'White Marvel'

(76) Inventor: Katsuya Sakaue, Takarazuka (JP)

Correspondence Address: Vincent G. Gioia, Christie, Parker & Hale, LLP, 5 Park Plaza, Suite 1440, Irvine, CA, 92614, US

(21) Appl. No.: 09/736,759

(22) Filed: Dec. 12, 2000

(51) Int. Cl. A01H 5/00

(52) U.S. Cl. PLT/226

(57) ABSTRACT

A new plant variety of Abelia×grandiflora having new shoots and foliage which emerge white, cream, pale yellow to pink to gold, with pink stems. Unusually thick mature foliage is golden to cream variegated with outer margins yellow green in the center of the foliage and bright yellow on the margins.

### BACKGROUND OF THE INVENTION

The present invention relates to a new and distinct variety of Abelia×grandiflora (André) Rehd., which was discovered as a branch sport in a controlled planting of Abelia ×grandiflora ‘Frances Mason’ in Takarazuka City, Japan by Mr. Katsuya Sakaue. The varietal denomination of the new variety is ‘White Marvel’.

The genus Abelia is included in the family Cáprifoliliaceae, which comprises about 16 genera and 365 species of herbs, shrubs, small trees and woody climbers, mostly temperate in origin. Abelia comprises approximately 15 species of deciduous orevergreen shrubs originating in China, Japan, Himalaya and Mexico, many of which possess desirable ornamental characteristics.

Abelia×grandiflora (André) Rehd. (Abelia chinensis×A. uniflora) is of unknown origin. It generally has opposite leaves, simple to ovate, 2-4 cm long and 1 to 2 cm wide. Leaves are acute or rounded or cuneate at base. Leaf margins are serrate, glabrous in surface texture. The plant is evergreen. Foliage color is dark green on the top surface, and pale green on the underside, depending on light.

Flowers of Abelia×grandiflora bloom May-June, and are approximately 2 cm wide.

### SUMMARY OF THE INVENTION

The new variety differs from its parent in having new shoots and foliage emerge white, cream, pale yellow to pink to gold, with pink stems. Mature foliage is golden to cream variegated with outer margins yellow green in the center of the foliage and bright yellow on the margins. Mature foliage is unusually thick. Asexual reproduction of the new variety by stem cuttings performed in Takarazuka City, Japan; Lewisberry, Pa. and Vacaville, Calif. have confirmed that the distinctive characteristics of the new variety are stable and transmitted to succeeding generations.

### COMPARISON WITH PARENTS

The new variety differs from its parent and all other varieties of Abelia×grandiflora known to the inventor in having new shoots and foliage emerge white, cream, pale yellow to pink, with pink stems. Mature foliage is golden variegated with outer margins yellow green in the center of the foliage and bright yellow on the margins.

### BRIEF DESCRIPTION OF THE ILLUSTRATIONS

The accompanying photographic illustrations show a specimen of the new cultivar as true to color as is reasonably possible to make in an illustration of this character.

In the illustrations:

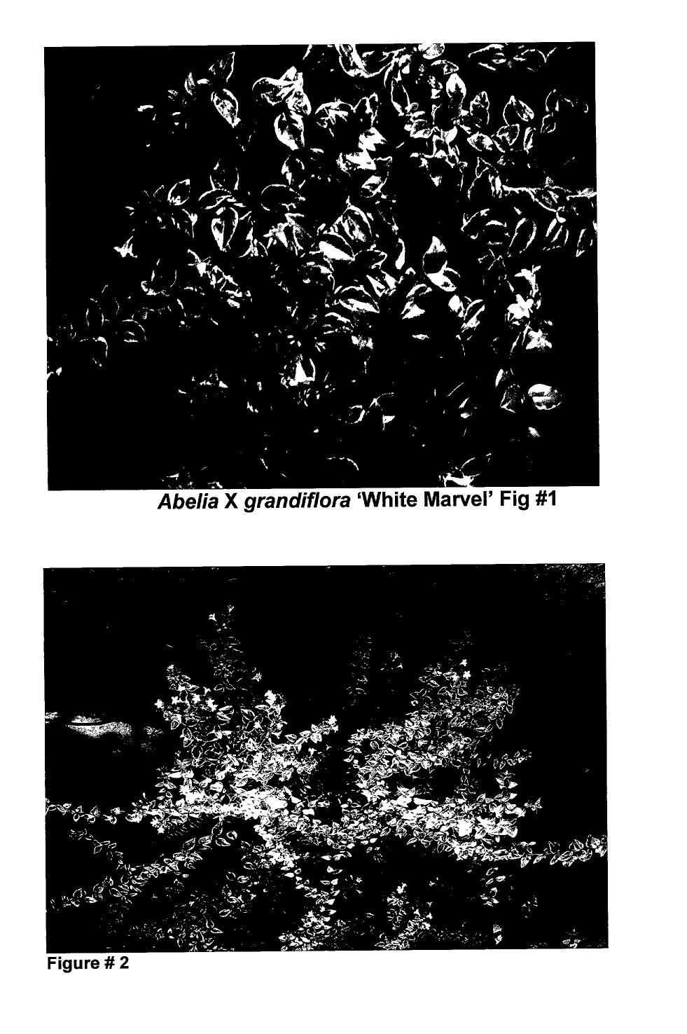

FIG. 1. illustrates new and secondary foliage prior to maturation.

FIG. 2. illustrates stems on the new growth as well as the newly emerged growth.

FIG. 3. illustrates secondary foliage with the distinct flecked/dotted cream to pale yellow variegation.

### DETAILED DESCRIPTION OF THE NEW VARIETY

‘White Marvel’ has not been observed under all possible environmental, cultural, and light conditions. The following observations and descriptions are of plants grown in containers at Lewisberry, Pa., and Vacaville, Calif. Phenotypic expression may vary with light intensity and cultural and environmental conditions. In this description, color references are to The Royal Horticultural Society Colour Chart (1995) and terminology used in the color descriptions herein refers to plate numbers in this color chart.

Classification:

Botanical.—Abelia×grandiflora ‘White Marvel’.

Parentage.—Branch sport of the variety Abelia×grandiflora ‘Frances Mason’.

Propagation.—By vegetative cuttings.

Plant:

Size.—Mature plant is 60 to 90 cm tall and wide.

Branching.—Branchlets opposite, puberulent.

Habit.—Evergreen flowering shrub.

Leaf shape.—Opposite, simple, ovate to ovate oval, acute or acuminate, cuneate at base.

Leaf margin.—Serrate.

Leaf texture.—Firm and glabrous above and below.

Leaf color:

NewGrowth:

Variegation leaf top.—Near Orange Red Group 31A.

Variegation leaf underside.—Near Orange Red Group 31A.

Mature Leaf, shaded by other foliage:

Leaf top Center.—Near Green Group 137C.

Variegation leaf top Margin.—Near Yellow-White Group 158B. Variegation margin is up to 3 mm wide.

Leaf bottom Center.—Near Green Group 1 37C.

Mature Leaf (full sun):

Leaf top Center.—Near Yellow Green Group 144A.

Variegation leaf top Margin.—Near Yellow Group 4D with occasional 151 B-D. Variegation margin is up to 3 mm wide.

Leaf bottom Center.—Same as top of foliage.

Petioles.—Approximately 4 mm long.

Leaf size.—1.5 to 4 cm long, 0.75 to 2 cm wide.

Hardiness.—USDA Zone 5 (−20° F. to 10° F.).

Infloresence:

Bloom Period.—Summer.

Flower Form.—Campanulate.

Flower Arrangement.—1 to 4, clustered in the leaf axils, sometimes terminal clusters or panicles.

Flower Size.—About 1 cm long and 1.5 cm wide.

Color.—White.

Shape of Inflorescence.—Funnel shaped.

Fragrance.—Sweet.

Reproductive organs:

Stamens.—4, inserted at base of corolla tube.

Stigma.—1, elongated.

Fruit.—1 seed, achene.

Sepalous Florets:

Number of sepals.—2 to 5.

Aspect of sepals.—Separate to partly connate, purplish.

Size of sepals.—3 to 5 mm long.

I claim:

1. A new plant variety of Abelia×grandiflora of the variety substantially as shown and described.
